(19) United States
(12) Patent Application Publication
(10) Pub. No.: US 2002/0192221 A1
(43) Pub. Date: Dec. 19, 2002

(54) METHOD FOR TREATING TUMOR USING A COMBINATION OF ENERGY AND ANTIBODY CONJUGATED TOXIN

(76) Inventor: Alfred J. Roach, Lindenhurst, NY (US)

Correspondence Address: PENNIE AND EDMONDS, 1155 AVENUE OF THE AMERICAS, NEW YORK, NY, 100362711

(21) Appl. No.: 09/838,008

(22) Filed: Apr. 19, 2001

(51) Int. Cl. A61K 39/395; A61N 7/00

(52) U.S. Cl. 424/178.1; 604/20

(57) ABSTRACT

The present invention relates to a method for treating a tumor that contains fibrin. The method comprises utilizing an energy from an energy source to destroy at least a portion of (i) a fibrin contained in the tumor or (ii) fibrin surrounded tumor. The energy may also cause necrosis of tumor cells that are adjacent to the fibrin. The method further include the use of a tumor-targeting monoclonal antibody conjugated to a toxin which is administered to the subject. The present invention also relates to a method for removing and killing fibrin-associated tumor cells from a mixed cell population, which method includes the step of delivering an energy from an energy source to the fibrin-associated tumor cells and killing the tumor cells with a tumor-targeting monoclonal antibody conjugated to a toxin.

### 1. FIELD OF THE INVENTION

The present invention relates to a method for treating a tumor. Specifically, the present invention relates to a method for treating a tumor that contains fibrin. In particular, the invention is directed to a method for treating a tumor that contains fibrin, which method comprises delivering an energy from an energy source and administering a tumor-targeting monoclonal antibody that is conjugated to a toxin. The present invention is also related to a method for removing and killing tumor cells from a mixed cell population.

### 2. BACKGROUND OF THE INVENTION

Tumor, especially cancer, is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis).

Pre-malignant abnormal cell growth is exemplified by hyperplasia, metaplasia, or most particularly, dysplasia (for review of such abnormal growth conditions, see Robbins & Angell, 1976, Basic Pathology, 2d Ed., W. B. Saunders Co., Philadelphia, pp. 68-79). The neoplastic lesion may evolve clonally and develop an increasing capacity for growth, metastasis, and heterogeneity, especially under conditions in which the neoplastic cells escape the host's immune surveillance (Roitt, I., Brostoff, J and Kale, D., 1993, Immunology, 3rd ed., Mosby, St. Louis, pps. 17.1-17.12).

Often, cancer is difficult to manage clinically. Clinical methods of treatment which are more effective and less invasive are needed. One of such less invasive methods is the use of a monoclonal antibody which is specific to a tumor antigen conjugated with a toxin.

Most of the solid malignant tumors and benign tumors contain fibrin. As Biggerstaff et al. describes, fibrin appears as a meshwork throughout tumor stroma (Biggerstaff et al., 1997, Cancer and Thrombosis, S (June) ppS492-S-492). The fibrins are often concentrated at the interface between the tumor and the normal tissue (see Honn et al., Hemostatic Mechanisms and Metastasis, Martinus Nijhoff, Boston, 1984, 96-114). Biggerstaff et al. also suggest that fibrin plays a role in metastasis, i.e., malignant spread (Biggerstaff et al., 1998, Thrombosis Research 92: S53-S58). Various reports suggest many functions of tumor-associated fibrin, such as, to promote tumor growth and neovascularization of growing tumor, and to aid tumor cell adhesion to endothelial cells during metastasis. Especially, there are some reports which show that fibrin protect tumors from immune or chemotherapeutic attack. Id. Therefore, a conventional tumor therapy using antibodies that target tumors may not effectively treat tumors that contain fibrin because the tumors are protected by the fibrin. Thus, a more effective method of treatment for tumors that contains fibrin is necessary.

Citation or identification of any reference herein shall not be construed as an admission that such reference is available as prior art to the present invention.

### 3. SUMMARY OF THE INVENTION

The present invention is based on finding that when tumor that contains fibrin is treated with an energy from an energy source, tumor-targeting monoclonal antibody that is conjugated to a toxin is more effective in treating the tumor.

The present invention is based upon the observation of the present inventor that fibrin exists in many tumors and that these tumors can be treated using various energy in combination with a tumor-targeting monoclonal antibody conjugated to a toxin.

The subject invention is aimed at providing a method for treating a tumor that contains fibrin in the body of a subject. The method comprises delivering an energy from an energy source, which includes, but not limited to, acoustic energy source, electromagnetic energy source or light energy source, to the tumor and administering to the subject a composition comprising tumor-targeting monoclonal antibody conjugated to a toxin.

The invention further provides a method for removing and killing tumor cells that associate with fibrin from a mixed cell population. The method comprises delivering an energy from an energy source, which includes, but not limited to, acoustic energy source, electromagnetic energy source or light energy source to the mixed cell population; and adding to the mixture a composition comprising a tumor-targeting monoclonal antibody conjugated to a toxin.

### 4. DETAILED DESCRIPTION OF THE INVENTION

In one embodiment, the method of the present invention comprises the delivery of an energy from an energy source and the administration to a subject, a composition containing a tumor-targeting monoclonal antibody which is conjugated to a toxin. Once an energy is delivered to a subject and the tumor-targeting monoclonal antibody which is conjugated to a toxin acts like a guided “smart bomb”. The tumor-targeting monoclonal antibody attaches to the fibrin or tumor antigen on the tumor and delivering the toxin to the tumor, thus destroying and/or neutralizing the tumor.

Traditionally, monoclonal antibodies used for cancer therapy are specific to antigens unique to tumor cells, i.e. tumor-specific monoclonal antibodies. Such tumor-specific monoclonal antibodies are explained in detail in section 4.3.1. Recently, however, the focus has been shifted to normal cells surrounding tumors and supporting their growth, such as tumor stroma and tumor vasculature (Welt et al., 1999, Seminars in Oncology, 26: 683-690). Specifically, a therapy using a fibrin-specific monoclonal antibody is disclosed in U.S. Pat. Nos. 5,453,359 and 5,871,737. The term “tumor-targeting monoclonal antibody” used here refers to either a fibrin-specific monoclonal antibody or a tumor-specific monoclonal antibody.

In the present invention, an energy from an energy source is delivered to the tumor to accelerate the process by which the tumor-targeting monoclonal antibody-conjugated toxin and the tumor interact. The energy from the energy source facilitates the action of the toxin from the tumor-targeting monoclonal antibody-conjugated toxin on the tumor cells. The energy used in the present method includes, but not limited to, acoustic energy, electromagnetic energy and light energy. Methods for treating tumors using various acoustic energy, electromagnetic energy and light energy have been employed for many years to treat tumors, thus avoiding an extensive surgery. Such energy includes, but not limited to, ultrasound waves, radio frequency and lasers. U.S. Pat. No. 5,873,828 discloses ultrasonic treatment system of cancers. U.S. Pat. Nos. 5,047,027, 5,928,229 and 6,132,425 discloses treatment devices using radio frequency energy. U.S. Pat. Nos. 4,822,335, 5,298,026 and 5,823,941 disclose treatment of tumor using lasers. However, most of these devices and methods use these energy for thermal therapy, i.e., heating a tumor to cause its necrosis. These devices and methods have not been in conjunction with a tumor-targeting monoclonal antibody conjugated to a toxin.

The method of the present invention may further comprise a step for which the tumor is removed by applying suction directly to the tumor to remove substantially the entire tumor, concurrently or after fragmenting the tumor mass by breaking the fibrin matrix by delivering energy from an energy source.

The method of the present invention may further comprise the step of conducting other tumor treatments such as surgery, radiation therapy or chemotherapy.

### 4.1 TUMOR THAT CONTAINS FIBRIN

The present invention is directed to a method for treating tumor that contains fibrin, which method comprises delivering an energy from an energy source and comprising administering a composition comprising a tumor-targeting monoclonal antibody conjugated with a toxin to the tumor.

“Tumor” includes, but is not limited to, neoplasms, cancer, metastasises, or any disease or disorder characterized by uncontrolled cell growth, and those diseases that can be treated by the method of the present invention. A tumor that contains fibrin may be in any form. The fibrin is usually deposited in the stroma within the tumor. The fibrin contained in a tumor may be non-crosslinked, partially or entirely crosslinked. Whether the method is effective to treat a certain type of tumor can be determined by any method known in the art, for example but not limited to, the methods as described in Section 4.4, infra.

### 4.1.1 TUMORS

The method of the present invention can be used to treat any tumor containing fibrin and tumor cells which can be identified by a cellular marker. The cellular marker can be any label that identifies and discriminates a tumor cell from a non-tumor cell. For example, tumor antigens and numerous tumor-specific markers have been found on a wide variety of tumorigenic cells.

Tumors which can be treated by the present method include, but not limited to, tumors listed in Table 1.

TABLE 1
Sarcomas and Carcinomas

fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon carcinoma
pancreatic cancer
breast cancer
ovarian cancer
prostate cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular tumor
lung carcinoma
small cell lung carcinoma
bladder carcinoma
epithelial carcinoma
glioma, glioblastoma
astrocytoma
medulloblastoma
craniopharyngioma
ependymoma
pinealoma
hemangioblastoma
acoustic neuroma
oligodendroglioma
meningioma
neuroblastoma
retinoblastoma
colorectal adnoma
malignant melanoma
uveal melanoma
primitive neuroectodermal tumor
papillary carcinoma of the thyroid
alveolar rhabdomyosarcoma
plemorphic adenoma of salivary glands
sporadic typical lipomas
extraskeletal nyxoidchondrosarcoma
mucoepidemoid carcinoma
adenolymphoma of salivary gland
intraabdominal desmoplastic small round cell tumor
askins tumor
ethesioneuroblastoma
uterine leiomyomas
myxoid liposarcoma

One embodiment of the method of the present invention encompasses the treatment of various types of tumors including, but not limited to, the tumors listed in Table 1. The tumor cells and/or tumor cell clusters in these tumors are surrounded by and/or connected to fibrin. Moreover, the method of the present invention can treat not only primary cancers but also metastatic cancers.

Another embodiment of the method of the present invention encompasses the treatment of fibroids, also known as leiomyoma, leiomyomata or fibromyoma, are the most common benign tumors of the uterus. Arising from the muscular wall of the uterus, their origin is thought to be the muscle in the walls of uterine blood vessels. In a specific embodiment, the method of the present invention encompasses the treatment of fibroid.

The method of the present invention can be used to treat any tumor that contains fibrin. Solid tumors may consist of tumor cells and/or tumor cell cluster which are connected to a structure called the stroma. Fibrin often deposits in the stroma. The fibrin may also surround at least a portion of the outer surface of a tumor. The method of the present invention is most useful, when a surgical resection is not proper, e.g., when the tumor is located in a dangerous area to be removed; when patient is not in a suboptimal condition for surgery, or when surgical resection is not the proper treatment. In addition, the method of the present invention can be used in combination with a surgical resection procedure. For example, the method of the present invention may be used prior to the surgical resection where the shrunken tumor is removed by the surgery. Alternatively, a tumor can be removed first by a surgical resection and then followed by treatment using the present invention. In an embodiment, the tumor may be removed by using suction applied directly to the tumor. The entire tumor mass may be substantially removed at one time. The suction can be conducted concurrently or after the energy delivery from an energy source to the tumor and also concurrently or after the administration of the composition containing a tumor-targeting monoclonal antibody conjugated to a toxin.

### 4.2 ENERGY DELIVERY TO TUMOR

An energy source is used in the method of the present invention. The energy source may include energy source such as, but not limited to, an acoustic energy source, an electromagnetic energy source and a light source. The energy may be delivered to the tumor for treatment prior to administration of a composition comprising a tumor-targeting monoclonal antibody conjugated to a toxin. In another embodiment of the present invention, the energy may be delivered concurrently with the administration of the composition. Other focused energy sources that are useful for the method of the present invention may include accelerated particles, electron beam or other radiator beam.

These energies delivered from energy sources fragment the tumor mass by breaking the fibrin matrix as sonication disrupt any protein by sonic/ultrasonic oscillation.

### 4.2.1 ENERGY AND ITS DELIVERY

An energy from an energy source is delivered to a tumor in a method of the present invention. Preferred energy sources are acoustic energy source, electromagnetic energy source and light source. The term “acoustic energy” means sonic energy or ultrasonic energy. An electromagnetic energy source may include, for example, radio frequency (“RF”) energy. A light energy source may include, for example, laser. For the method of the present invention, any device including probes and catheters and method suitable for delivering such energy to a soft tissue in a subject may be used.

In one embodiment of the present invention, a device may be used for the delivery of an energy from an energy source. The device may further comprise a suction probe which is connected to a suction pump. Alternatively, a suction probe may be a separate device from the device for energy delivery. In one embodiment, after a tumor is treated with an energy from an energy source and a composition comprising a monoclonal antibody conjugated to a toxin is administered, substantially the entire mass of the tumor may be removed using the suction probe. In another embodiment, a tumor may be removed using the suction probe while concurrently treated with an energy from an energy source and administration of a composition comprising a tumor-targeting monoclonal antibody conjugated to a toxin. In one embodiment, the tumor may be removed by suction after treatment with an energy source and before the administration of the composition comprising a tumor-targeting monoclonal antibody.

### 4.2.1.1 SONIC AND ULTRASONIC ENERGY

Ultrasound is well known in the medical field for its ability for tissue imaging, which provides an image of a cross-section of a tissue without the need to slice the tissue. Also, in broader field, sonication is used to disrupt proteins by sonic/ultrasonic oscillation. The present invention is aimed at exploiting this effect for the treatment of tumor. The key is to create a strong ultrasound intensity at the tumor mass to break the fibrin matrix while minimizing the ultrasound intensity in normal tissue.

Various methods and devices may be used in connection with the present invention. For example, U.S. Pat. No. 5,895,356 discloses a probe for transurethrally applying focused ultrasound energy to produce hyperthermal and thermotherapeutic effect in diseased tissue. U.S. Pat. No. 5,873,828 discloses a device having an ultrasonic vibrator with either a microwave or radio frequency probe. U.S. Pat. No. 6,056,735 discloses an ultrasonic treating device having a probe connected to a ultrasonic transducer and a holding means to clamp a tissue. Any of those methods and devices can be adapted for use in the method of the present invention.

Ultrasound ablation may be performed through an open incision or via laparoscopy which is performed through multiple, small skin incisions. Ultrasound ablation can also be conducted percutaneously through small skin incisions. An ultrasonic vibrator or probe can be inserted into a subject's body through a body lumen, such as blood vessels, bronchus, urethral tract, digestive tract, and vagina. However, an ultrasound probe can be appropriately modified, as known in the art, for subcutaneous application. The probe can be positioned closely or in contact to an outer surface of the tumor or can penetrate the surface of the tumor. The duration of the procedure depends on many factors, including the number of applications desired and the location of the tissue to be treated. Typically, the procedure will be performed in a surgical suite where the patient can be monitored by imaging equipment. Also, a plurality of probes can be used simultaneously in order to ablate more than one portion in the tumor during a single procedure. For example, a set of probes can be arranged as an umbrella.

For the purpose of the present invention, the sonic energy is defined as pressure wave that propagate through air or other media having frequency from about 1 kHz and about 16 kHz, and the ultrasonic energy is defined as sound waves of about 16 kHz or more. In general, the sound energy used for the present invention is between about 1 kHz and about 16 kHz. The ultrasound energy used for the present invention is between about 16 kHz and about 2 MHz, preferably between about 20 kHz and about 1 MHz. The ultrasound power (W; watt) to be emitted from the probe should be determined based on the frequency of the ultrasound, the area of probe emitting the ultrasound, the distance between the ultrasound probe and the focal plane, i.e., the tumor to be treated, and the energy needed at the tumor to be treated. In general, ultrasound energy of from about 1 to about 10 watts/cm² is delivered to the focal point of the tumor. The energy delivery cannot continue if the temperature of the tumor and of the surrounding area become too high. The method of the present invention may optionally use a cooling system. One skilled in the art can determine the proper cycle of the ultrasound, proper intensity of the ultrasound, and time to be delivered in each specific case based on experiments using an animal as a model.

### 4.2.1.2 RADIO FREQUENCY (RF)

Radio frequency energy has been used in medical procedures for many years for the treatment of soft tissue. Radio frequency ablation occurs from a high frequency alternating current flowing from the tip of an electrode through the surrounding tissue. Ionic agitation is produced in the tissue around the electrode tip as the ions attempt to follow the change in direction of the alternating current. This ionic agitation creates frictional heating of the tissue around the electrode. Thus, similar to a microwave, the tissue is being heated even though the electrode does not; although conducted heat may progress back to the electrode from the surrounding tissue. The heat generated results in ablation of the tissue and fragment the tumor mass.

Various methods and devices to deliver RF have been proposed. For example, as a device and/or a method of using radio frequency (“RF”), U.S. Pat. No. 6,132,425 discloses a cell necrosis apparatus having a flexible introducer which introduces RF probes into a target tissue. U.S. Pat. No. 5,928,229 discloses an ablation apparatus having plurality of RF antennas. U.S. Pat. No. 5,762,626 discloses a transurethral needle ablation device using RF. Any of those methods and devices can be adapted for use in the method of the present invention.

RF ablation may be performed through an open incision or via laparoscopy which is performed through multiple, small skin incisions. If appropriate, RF ablation can also be conducted percutaneously through small skin incisions. A RF probe can be inserted into a subject's body through a body lumen, such as blood vessels, bronchus, urethral tract, digestive tract, and vagina. The probe can be positioned closely or in contact to an outer surface of the tumor or can penetrate the surface of the tumor. The duration of the procedure depends on many factors, including the number of applications desired and the location of the tissue to be treated. Typically, the procedure will be performed in a surgical suite where the patient can be monitored by imaging equipment. Also, a plurality of probes can be used simultaneously in order to ablate more than one point in the tumor in one procedure. For example, a device that is currently on the market, the “LeVeen Needle Electrode” by Radio Therapeutics Corporation, deploys an umbrella of 10 tines which burrow into the tumor growth distributing the RF energy thereby heating and destroying the cells. Following the procedure, the body reabsorbs the destroyed cells over a period of time. Structurally, the LeVeen Needle Electrode consists of ten evenly spaced wires that are deployed through an insulated metal cannula (U.S. Pat. Nos. 5,827,276, 5,855,576, and 5,868,740). When deployed, the array of wires advances through the tissue in a constant radius curve away from the metal cannula, producing the umbrella shaped design of the LeVeen Needle Electrode. The cannula supports the array which is deployed within its predetermined shape and dimensions which allows penetration of the tissue irrespective of density. Thus, the wires of the LeVeen Needle Electrode can be deployed into tissue which is hard, such as calcified tissue in its entirety.

In general, the RF energy used for the present invention is preferably between about 50 kHz and about 50 MHz, and more preferably between about 500 kHz and about 10 MHz. The RF power (W; watt) to be emitted on a tumor is over about 5 watts/cm² is delivered to the focal point of the tumor. The energy delivery cannot continue if the temperature of the tumor and of the surrounding area become too high. The method of the present invention may optionally use a cooling system. One skilled in the art can determine the proper cycle of the RF, proper intensity of the RF, and time to be delivered in each specific case based on experiments using an animal model.

### 4.2.1.3 LIGHT ENERGY

One of the energy source that can be used in the method of the present invention is light energy source. A controlled energy source, such as a laser, collimated or focused non-laser light, infrared light or ultraviolet light may be used in the present invention. The term “laser” is an acronym for light amplification by stimulated emission of radiation. Laser has been used for many years in medical procedure. Laser ablation occurs from a stimulated coherent emission of photons, i.e., laser beam, from the tip of an optical fiber through the surrounding tissue.

Various methods and devices to deliver laser may be used in the method of the present invention. For example, U.S. Pat. No. 5,298,026 discloses an apparatus and a method treating tumor by applying laser through optical fibers. U.S. Pat. No. 5,823,941 discloses an apparatus for removing brain tumor by using a laser beam. U.S. Pat. No. 4,822,335 discloses an apparatus for treating a tumor by irradiating light energy.

Laser ablation may be performed through an open incision or via laparoscopy which is performed through multiple, small skin incisions. Laser ablation can also be conducted percutaneously through small skin incisions. The optical fiber can be inserted into a subject's body through a body lumen, such as blood vessels, bronchus, urethral tract, digestive tract, and vagina. However, the optical fiber can be appropriately modified, as known in the art, for subcutaneous (through skin) application. The fiber can be positioned closely to or in contact with the outer surface of a tumor or can penetrate the surface of the tumor. The duration of the procedure depends on many factors, including the number of applications desired and the location of the tissue to be treated. Typically, the procedure will be performed in a surgical suite where the subject can be monitored by imaging equipment. Also, a plurality of probes can be used simultaneously in order to ablate more than one portion of the tumor during a single procedure. For example, the probe for the delivery of laser may have a set of applicators that can be arranged as an umbrella.

In general, the laser used for the present invention has power range that is higher than about 5 watts, and preferably between about 10 to 500 watts. A preferred wavelengths of light in the range of 375 to 900 nanometers. One such laser is made by Photonic Instruments (Arlington Heights, Ill.). As is known, lasers provide a monochromatic light source with highly specific directionality, making it possible to control a focused beam of energy. High precision, both in terms of spatial and temporal resolution, in the delivery of light energy can be achieved at predefined wavelengths. The most advantageous wavelength that can be used in the method of the present invention can be determined experimentally and employed for the most advantageous results. As an alternative, broad-band light sources (e.g. as obtained from a Xenon Lamp) can be employed in the method of the invention to provide a controlled energy source. The power of the light source, and the duration of the light pulse, can be adjusted to achieve the desired result. A laser that can be used for the present invention may be of various types including, but not limited to, Argon laser, CO₂ laser and nedymium-YAG laser. Laser pulse lengths can be as short as 2 to 6 nanoseconds, or an ultrashort-pulse laser which is laser that consists of pulses with durations shorter than about 10 pico (=10⁻¹¹) second. The light energy can be emitted at up to 50 microjoules or more. However, the total amount of light energy provided to the designated tumor is preferably selected not to cause substantial boiling of the targeted cellular material or the surrounding medium.

The local heating of cellular medium to the point of boiling can damage normal non-tumor cells. In one embodiment, the total light power delivered will minimally disrupt the cellular membrane or cellular components but cause damage to the stroma or a meshwork that consists of fibrin. Alternatively, the total light power can be chosen to cause eventual tumor necrosis and cause damage to the stroma or a meshwork consisting of fibrin.

The method of the present invention may optionally use a cooling system and or a suction for suctioning out the gases which may be produced by the application of laser energy. One skilled in the art can determine the proper laser wave length, proper intensity of the laser, and time to be delivered in each specific case based on experiments using an animal model.

### 4.3 COMPOSITION CONTAINING TUMOR-TARGETING MONOCLONAL ANTIBODIES

In this specification, the term a “tumor-targeting monoclonal antibody” means a monoclonal antibody that is specific to a cellular marker that identifies and discriminates a tumor cell from a non-tumor cell, such as a tumor-specific monoclonal antibody and a fibrin-specific monoclonal antibody.

### 4.3.1 TUMOR-SPECIFIC MONOCLONAL ANTIBODY

A tumor-specific monoclonal antibody is a monoclonal antibody that is specific to a tumor antigen. A lot of tumor antigens have been found on a wide variety of tumorigenic cells.

Tumor-specific antigens or immunoactive fragments or derivatives thereof can be used to generate a tumor-specific monoclonal antibody that is used in the method of the present invention. In the present invention, the term “tumor-specific antibody” includes a monoclonal antibody that is specific to a tumor antigen or immunoactive fragments or derivative thereof and immunoactive fragments or derivatives of the antibody that competitively inhibits the immunospecific binding of the monoclonal antibody to its target antigen. A composition containing a monoclonal antibody or fragment or derivative of the monoclonal antibody which is conjugated to a toxin is administered to a subject.

A method for preparing a monoclonal antibody against an antigen is well established. For example, but not by way of limitation, the monoclonal antibody used in the present invention include monoclonal antibodies specific to the following tumor antigens: KS ¼ pan-carcinoma antigen (Perez and Walker, 1990, J. Immunol. 142:3662-3667; Bumal, 1988, Hybridoma 7(4):407-415), ovarian carcinoma antigen (CA125) (Yu et al., 1991, Cancer Res. 51(2):468-475), prostatic acid phosphate (Tailor et al., 1990, Nucl. Acids Res. 18(16):4928), prostate specific antigen (Henttu and Vihko, 1989, Biochem. Biophys. Res. Comm. 160(2):903-910; Israeli et al., 1993, Cancer Res. 53:227-230), melanoma-associated antigen p97 (Estin et al., 1989, J. Natl. Cancer Instit. 81(6):445-446), melanoma antigen gp75 (Vijayasardahl et al., 1990, J. Exp. Med. 171(4):1375-1380), high molecular weight melanoma antigen (HMW-MAA) (Natali et al., 1987, Cancer 59:55-63; Mittelman et al., 1990, J. Clin. Invest. 86:2136-2144), prostate specific membrane antigen, carcinoembryonic antigen (CEA) (Foon et al., 1994, Proc. Am. Soc. Clin. Oncol. 13:294), polymorphic epithelial mucin antigen, human milk fat globule antigen, colorectal tumor-associated antigens such as: CEA, TAG-72 (Yokata et al., 1992, Cancer Res. 52:3402-3408), CO17-1A (Ragnhammar et al., 1993, Int. J. Cancer 53:751-758); GICA 19-9 (Herlyn et al., 1982, J. Clin. Immunol. 2:135), CTA-1 and LEA, Burkitt's lymphoma antigen-38.13, CD19 (Ghetie et al., 1994, Blood 83:1329-1336), human B-lymphoma antigen-CD20 (Reff et al., 1994, Blood 83:435-445), CD33 (Sgouros et al., 1993, J. Nucl. Med. 34:422-430), melanoma specific antigens such as ganglioside GD2 (Saleh et al., 1993, J.Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250), tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chatterjee et al., 1988, J. of Immunospecifically. 141:1398-1403), neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57), primary endoderm, preimplantation embryos, I(Ma) found in gastric adenocarcinomas, M18, M39 found in breast epithelium, SSEA-1 found in myeloid cells, VEP8, VEP9, Myl, VIM-D5, D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, G_{M2}, OFA-2, G_{D2}, and M1:22:25:8 found in embryonal carcinoma cells, and SSEA-3 and SSEA-4 found in 4 to 8-cell stage embryos.

A composition containing a monoclonal antibody or fragment or derivative of the monoclonal antibody which is conjugated to a toxin is used in the method of the present invention. The fragment is a functionally active fragment of a monoclonal antibody. Functionally active fragment means that the fragment can immunospecifically bind the target antigen as determined by any method known in the art to determine immunospecific binding. For example, such fragments include but not limited to: F(ab′)₂ fragments, which contain the variable regions of both the heavy and the light chains, the light constant region and the CH1 domain of the heavy chain, which fragments can be generated by pepsin digestion of the antibody, and the Fab fragments, generated by reducing the disulfide bonds of an F(ab′)₂ fragment (FIG. 1; King et., 1992, Biochem. J. 281:317); and Fv fragments, i.e., fragments that contain the variable region domains of both the heavy and light chains (Reichmann and Winter, 1988, J. Mol. Biol. 203:825; King et al., 1993, Biochem. J. 290:723).

Single chain antibodies may also be used in the method of the present invention (SCA) (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546). Single chain antibodies are formed by linking the heavy and light chain fragments of the Fv region via an amino acid bridge, resulting in a single chain polypeptide. Additionally, the heavy chain and light chain dimers and diabodies may also be used in the method of the present invention.

Chimeric or humanized antibodies may also be used in the method of the present invention. A chimeric antibody is a molecule in which different portions of the antibody molecule are derived from different animal species, such as those having a variable region derived from a murine mAb and a constant region derived from a human immunoglobulin constant region, e.g., humanized antibodies. Techniques have been developed for the production of chimeric antibodies (Morrison et al., 1984, Proc. Natl. Acad. Sci. 81:6851-6855; Neuberger et al., 1984, Nature 312:604-608; Takeda et al., 1985, Nature 314:452-454; International Patent Application No. PCT/GB85/00392 (Neuberger et al. and Celltech Limited)) by splicing the genes from a mouse antibody molecule of appropriate antigen specificity together with genes from a human antibody molecule of appropriate biological activity can be used.

The antibodies that may be used in the method of the present invention may be modified, e.g, by the covalent attachment of any type of molecule, as long as such covalent attachment does not prevent or inhibit immunospecific binding of the immunoglobulin to its target antigen. For example, but not by way of limitation, the antibodies may be modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc.

Specifically, the antibodies that are used in the method of the present invention maybe covalently linked to a toxin to target the toxin to a tumor. The toxin can be any type of therapeutic molecule known in the art, for example, but not limited to, a chemotherapeutic agent, a toxin, such as ricin, an antisense oligonucleotide, a radionuclide, etc. which is discussed in section 4.3.3, infra.

### 4.3.2 FIBRIN-SPECIFIC MONOCLONAL ANTIBODY

Since fibrin appears as a meshwork throughout tumor stroma and is necessary for tumor growth, a fibrin-specific monoclonal antibody can be used for treatment of tumor in the present invention.

A fibrin-specific monoclonal antibody can be prepared using any conventional way for preparing monoclonal antibody. One example for such fibrin-specific monoclonal antibodies is MH1 disclosed in U.S. Pat. Nos. 5,453,359 and 5,871,737. The fibrin-specific monoclonal antibody used in the present invention may be a monoclonal antibody that competitively inhibits the immunospecific binding of the MH1 antibody produced by hybridoma ATCC HB 9739 to MH1's target antigen, wherein the monoclonal antibody does not crossreact with: (a) fibrinogen, (b) plasmin derived fibrinogen degradation products and (c) plasmin derived fibrin degradation products.

All the descriptions made above in section 4.3.1 as to a functionally active fragment, single chain, chimeric or humanized antibodies and modified antibody in relation to a tumor-specific monoclonal antibody are also applicable to a fibrin-specific monoclonal antibody.

### 4.3.3 MONOCLONAL ANTIBODY CONJUGATES

In the method of the present invention, a composition which comprises a tumor-targeting monoclonal antibody conjugated to a toxin is used. Examples for such fibrin-specific monoclonal antibody conjugated to a toxin are disclosed in U.S. Pat. Nos. 5,871,737 and 6,187,593. The monoclonal antibody molecule may be conjugated to the toxin via non-covalent or covalent linkages. Since non-covalent bonds are more likely to be broken before the antibody-toxin reagent complex reaches the target site, covalent linkages are preferred. For instance, a carbodiimide bond can be formed between the carboxy groups of the toxin and the amino groups of the monoclonal antibody molecule. Bifunctional agents such as dialdehydes or imidoesters can be used to link the amino group of a drug to amino groups of the antibody molecule.

The Schiff base reaction can be used to link antibody molecules to a toxin. This method involves the periodate oxidation of a toxin that contains a glycol or hydroxy group, thus forming an aldehyde which is then reacted with the antibody molecule. Attachment occurs via formation of a Schiff base with amino groups of the antibody molecule. Additionally, toxins with reactive sulfhydryl groups can be coupled to antibody molecules.

Many kinds of toxins are well know in the art and used for tumor therapy. More than one kind of toxins can be used to conjugate with the monoclonal antibody.

A toxin that can be conjugated to a monoclonal antibody for use in the method of the present invention includes any reagent that is effective to kill tumor cells (cytotoxin) or to arrest tumor cell growth (therapeutic agent). Toxins that can be used in the present invention include, but not limited to, taxol, thioguanine, hydroxyurea, cytarabine, cyclophosphamide (such as 4-Hydroxy-peroxy-cyclo-phosphamide(4Hc)), ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, cytochalasin B, gramicidin D, ethidium bromide, emetine, mitomycin, etoposide, tenoposide, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol,puromycin a ricin toxin, and a radionuclide and analogs or homologs thereof. Toxins may also include, but not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

### 4.3.4 COMPOSITION

Compositions containing a tumor-targeting monoclonal antibody conjugated to a toxin for use in accordance with the method of the present invention can be formulated in any conventional manner using one or more physiologically acceptable carrier or excipients. A pharmaceutically acceptable additive and/or diluent may be contained in the composition.

The composition can be formulated for parenteral administration (i.e., intravenous, subcutaneous or intramuscular) by injection, via, for example, bolus injection or continuous infusion.

Generally, the ingredients for the composition of the present invention are supplied either separately or mixed together in unit dosage form, for example, as a dry lyophilized powder or water free concentrate in a hermetically sealed container such as an ampoule or sachette indicating the quantity of active agent. An ampoule of sterile diluent can be provided so that the ingredients may be mixed prior to administration. The compositions can take such forms as suspensions, solutions in oily or aqueous vehicles, and can contain formulatory agent such as suspending, stabilizing and/or dispersing agents. Alternatively, the active ingredient can be in powder form for constitution with a suitable vehicle, e.g., sterile pyrogen-free water, before use. Each tumor-targeting monoclonal antibody that is conjugated to a toxin may be administered as separate composition or as a single composition with more than one antibodies conjugated to toxin complex linked by conventional chemical or by molecular biological methods.

The invention also provides a pharmaceutical pack or kit comprising one or more containers filled with one or more of the ingredients of the composition used in the method of the present invention. Associated with such container(s) can be a notice in the form prescribed by a governmental agency regulating the manufacture, use or sale of pharmaceuticals or biological products, which notice reflects approval by the agency of manufacture, use or sale for human or veterinary administration.

### 4.3.5 METHODS OF ADMINISTRATION

Different techniques of administration may be employed for the introduction of the composition used in the method of the present invention. One skilled in the art can determine the most desirable way of administering the composition depending on the type of treatment and condition of the subject. These include but not limited to intravenous, percutaneous or any other standard routes of administration. When a probe or catheter is used to deliver an energy from an energy source, such probe or catheter can provide a lumen for introducing the composition directly to the tumor. The placement of the probe or catheter in relation to the tumor may depend on the viscosity of the composition, the location of the tumor to be treated or the size of the vessel in which the delivery probe or catheter is placed.

The method of the present invention can also be used in various liquid-filled regions of the body, such as the lymphatic, digestive, urinary, reproductive, biliary and other systems, as well as inter-peritoneal, intra-cranial, intra-thoracic and other body cavities and spaces.

### 4.3.6 EFFECTIVE DOSE

The precise dose of the composition used in the method of the present invention to be employed will depend on the size of the tumor to be treated, the nature of the tumor, and should be decided according to the judgment of the practitioner and each patient's circumstances according to standard clinical techniques. An effective amount is that amount sufficient to produce tumor necrosis or to stop tumor growth. Effective doses may also be extrapolated from dose-response curves derived from animal model test systems.

Toxicity and therapeutic efficacy of composition used in the method of the present invention can be determined by standard pharmaceutical procedures in experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions which exhibit large therapeutic indices are preferred. While compositions that exhibit toxic side effects can be used, care should be taken to design a delivery system that targets such composition to the vessel or duct in order to minimize potential damage to normal cells and, thereby, reduce side effects.

The data obtained from animal studies can be used in formulating a range of dosage for use in humans. The dosage of such compositions lies preferably within a range of concentrations that include the ED₅₀ with little or no toxicity. The dosage can vary within this range depending upon the dosage form employed. Such information can be used to more accurately determine useful doses in humans.

The optimal dosage to be administered will be readily determined by those skilled in the art and will vary on the condition being treated, the particular type of toxin and monoclonal antibody used and mode of administration. Other factors include the weight and condition of the human or animal. It is to be understood that the present invention has application for both human and veterinary use.

### 4.4 ASSESSMENT OF EFFICACY OF TREATMENTS

Whether a particular treatment of the invention is effective to treat a certain type of tumor can be determined by any method known in the art, for example but not limited to, those methods described in this section.

The safety and efficiency of the proposed method for treating a tumor and a composition used in the method may be tested in the course of systematic medical and biological assays on animals, toxicological analyses for acute and systemic toxicity, histological studies and functional examinations, and clinical evaluation of patients having a variety of indications for tumor.

The efficacy of the method of the present invention may be tested in appropriate animal models, and in human clinical trials, by any method known in the art. For example, the animal or human subject may be evaluated for any indicator of disease that the method of the present invention is intended to treat. Then, the efficacy of the method of the present invention for tumor treatment can be assessed by measuring the size of a tumor in the animal model or human subject at suitable time intervals before, during, or after the treatment. Any change or absence of change in the size of the tumor can be identified and correlated with the effect of the treatment on the subject. The size of the tumor can be determined by any method known in the art (e.g., by ultrasound). Other improvements can be determined by any method known in the art.

In fibroid treatment, the patient may undergo transabdominal and endovaginal pelvic ultrasound examination prior to the treatment as well as after the treatment. Longitudinal, anteroposterior, and transverse measurements of the uterus and dominant fibroid may be obtained. Approximate uterine volume and volume of dominant fibroid can be calculated with use of the formula for a prolate ellipsoid, as previously described (Onisini et al, 1984, Radiology 153:113-116). Percent volume reduction can be calculated for each patient, and statistical comparison of pre-treatment and post-treatment uterine volumes can be accomplished (paired t test). Other symptoms may likewise be monitored, including but not limited to, abnormal bleeding, pelvic pain, abdominal or pelvic fullness, pressure in the colon and urinary bladder, causing constipation or frequent urination, and leg swelling etc.

Likewise, the progress of a cancer or tumor before and after the treatment may be monitored by assaying the levels of the particular cancer or tumor antigen (or another antigen associated with the particular cancer or tumor) either in the serum of the subject. Additionally, other imaging techniques, such as computer tomographic (CT) scan, sonograms, or any other imaging method, may be used to monitor the progression of the cancer or tumor both before and after the treatment. Biopsies may also be performed. Before carrying out such trials in humans, the tests for efficacy of the method of the invention can be performed in animal models of the particular cancer or tumor.

### 4.4.1 ANIMAL MODEL EXPERIMENTS

Examples of animal model to assess efficacy of the method of the present invention are described below. Tumor cells used in the experiment should be chosen according to the tumor to be treated.

### EXAMPLE 1-1 (melanoma)

Female rabbits of 6-8 weeks old are obtained. Rabbits, in groups of five or ten, are each given a subcutaneous injection on the saved flank a 5×10⁶ A375 cells (melanoma cells). Tumor size is assessed by measuring two perpendicular diameters in millimeters (mm) using a calliper weekly for each animal. The results are expressed as mean diameter of tumors.

When the tumor size reaches 10 mm, some rabits are treated with ultrasound from an ultrasound source delivered through a probe to the tumor. The intensity, cycles and/or time of the ultrasound delivery may be changed in different experimental groups.

A fibrin-specific monoclonal antibody MH1 is conjugated to cisplatin and suspended in a physiological salt solution to make a 10% MH1 conjugate suspension. Immediately after delivery of the ultrasound, about 1 ml of the suspension is subcutaneously injected into the tumor of rabits of every group. If desired, concentration of the MH1 conjugate can be changed in different experimental groups.

Tumor size are measured daily for two (2) weeks.

### EXAMPLE 1-2

Example 1-2 is identical to Example 1-1 except that when the tumor size reaches 10 mm, some rabits are treated with RF from an electromagnetic energy source delivered through a probe to the tumor. The power, cycles and/or time of the RF delivery may be changed in different experimental groups.

### EXAMPLE 1-3

Example 1-3 is identical to Example 1-1 except that when the tumor size reaches 10 mm, some rabits are treated with laser beam delivered through a probe to the tumor. The power, pulse length and/or time of the laser application may be changed in different experimental groups.

### EXAMPLE 2-1 (fibrosarcoma)

Example 2-1 is identical to Example 1-1 except that rabbits are that are 4-8 week old and fibrosarcoma cells NCTC2555 are injected subcutaneously to form a tumor.

### EXAMPLE 2-2

Example 2-2 is identical to Example 2-1 except that when the tumor size reaches 10 mm, some rabits are treated with RF from an electromagnetic energy source delivered through a probe to the tumor. The power, cycles and/or time of the RF delivery may be changed in different experimental groups.

### EXAMPLE 2-3

Example 2-3 is identical to Example 2-1 except that when the tumor size reaches 10 mm, some ragits are treated with laser beam delivered through a probe to the tumor. The power, pulse length and/or time of the laser application may be changed in different experimental groups.

### 4.5 TUMOR DETECTION

The method of the present invention may further comprise detection of the tumor of interest located and/or measured before an energy from an energy source is delivered or a composition comprising a tumor-targeting monoclonal antibody conjugated to a toxin is administered. There are many techniques to detect tumors. One skilled in the art may select different techniques may be used to detect the tumor of interest. For example, such decision may be made based on the kind of tumor and the subject's conditions. Methods which may be used include detecting the transmission characteristics of electromagnetic and sound (elastic wave) radiation. These include medical ultrasonic imaging (both transmission and echographic), X-ray imaging (e.g. CT scanning), nuclear magnetic resonance (NMR) imaging, magnetic resonance image (MRI) and microwave imaging.

The detection step in the method of the present invention may involve administering a composition comprising a tumor-targeting monoclonal antibody that is conjugated to a label. The label enables visualization of the tumor, and allows the surgeon to monitor the procedure while it is being performed. Examples of a label include, but not limited to, various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. One skilled in the art can determine the suitable type of label to be used and how to conjugate the monoclonal antibody to the label. The type of conjugation described in Section 4.3.3 supra can be used.

In accordance to a specific embodiment of the method of the present invention, the composition used in the method of the present invention is introduced into the blood vessel, generally by injection, catheterization or the like as described in Section 4.3.5 supra. The label is then detected by any conventional methods known by one skilled in the art. In general, one of skill in the art would determine the method of visualization depending on the type of label, reagent or composition used in the method of the present invention (For a review, see Zalcberg, J. R., (1985) Am. J. Clin. Oncl. 8:481-9, and Carrasquillo, J. A., et al., 1984, Cancer Treatment Reports 68:317-28).

### 4.6 METHOD FOR REMOVING AND KILLING TUMOR CELLS THAT ASSOCIATED WITH FIBRIN FROM A MIXED CELL POPULATION

Hematopoietic stem cell transplantation is a rapidly growing therapy throughout the world. Hematopoietic stem cells are cells that reside in the bone marrow which are responsible for production of all of the body's blood cells. In 1995, over twenty thousand hematopoietic stem cell transplants were performed in the United States. In particular, the treatment of breast cancer with autologous hematopoietic stem cell transplantation has become a widely used cancer therapy.

Tumor metastasis is a well-known process by which tumor cells leave their initial location and spread to other parts of the body. Once transported to a new site, the tumor cells begin to grow and populate the new site, thus creating a new tumor. One treatment for patients with metastatic tumors involves harvesting their hematopoietic stem cells and then treating the patient with high doses of radiotherapy or chemotherapy. This treatment is designed to destroy all the patients tumor cells, but has the side effect of also destroying their hematopoietic cells. Thus, once the patient has been treated, the autologous stem cells are returned to their body. However, if the tumor cells have metastasized away from the tumor's primary site, there is a high probability that some tumor cells will contaminate the harvested hematopoietic cell population. In such a case, the harvested hematopoietic stem cells include contaminating tumor cells. It is important to provide a method for killing all of the metastasized tumor cells prior to reintroducing the stem cells to the patient. If any living tumorigenic cells are re-introduced into the patient, they can lead to a relapse.

The problem of removing tumor cells from hematopoietic cells has been reported during traditional bone marrow harvest procedures (Campana et al. 1995, Blood, 85(6): 1416-34). The number of contaminating tumor cells ranged from about 10 to 5000 tumor cells per four million mononuclear harvested cells, depending on the chemotherapeutic drug regimen used for mobilization. Thus, a great need exists for efficient methods for removing all of the tumor cells from a hematopoietic cell transplant (Gulati et al, 1993 2(4):467-71) (U.S. Pat. No. 6,143,535). Accordingly, the present invention provides a rapid and reliable method for removing all of the contaminating tumor cells so as to improve the efficacy of hematopoietic stem cell transplantation.

In one specific embodiment, the method relates to eliminating tumor cells that associate with fibrin from a population of non-tumor cells. In particular, this aspect of the present invention relates to a method for destroying tumor cells by delivering an energy from an energy from an energy source and administering a composition containing a tumor-targeting monoclonal antibody conjugated to a toxin. In the present invention, an energy from an energy source is delivered concurrently or before the administration of the composition. The energy from the energy source and facilitate the action of toxin on the tumor cells.

Accordingly, one embodiment of the present invention provides a method of eliminating tumor cells from a population of cells that includes non-tumor cells that do not associate with fibrin, including the steps of: a) applying an energy from an energy source in a mixed cell population comprising fibrin-associated cells; b) killing the fibrin-associated tumor cells with a tumor-targeting monoclonal antibody that is conjugated to a toxin.

In another embodiment, the method provides for enriching the number of stem cells in a hematopoietic cell population, including: a) applying an energy from an energy source to a mixed cell population comprising fibrin-associated tumor cells; b) killing the fibrin-associated tumor cells with a tumor-targeting monoclonal antibody that is conjugated to a toxin.

Still another embodiment is a method for preparing isolated hematopoietic cells for re-introduction into a patient, including: a) applying an energy from an energy source to a mixed cell population comprising fibrin-associated tumor cells; b) killing the fibrin-associated tumor cells with a tumor-targeting monoclonal antibody that is conjugated to a toxin.

In another embodiment, individual tumor cell that associates with fibrin are identified using a tumor-targeting monoclonal antibody that is conjugated to a label. The detected cell is specifically killed with energy from an energy source. In another embodiment, the detected cell is killed using a tumor-targeting monoclonal antibody that is conjugated to a toxin. In a specific embodiment, the tumor-targeting monoclonal antibody is fibrin specific monoclonal antibody. In another specific embodiment, the tumor-targeting monoclonal antibody is specific to tumor antigen. In a preferred embodiment, the toxin is 4-hydroxy-peroxy-cyclo-phosphamide (4HC).

The energy delivered to the cell population can be either sonic, ultrasonic, radio frequency, or laser, as described in Section 4.2, supra. One skilled in the art can use a proper intensity and time of radiation so as not to damage the normal cells. The composition containing a tumor-targeting monoclonal antibody that is conjugated to a toxin is described in Section 4.3, supra.

The description contained herein is for purposes of illustration and not for purposes of limitation. Changes and modifications may be made to the embodiments of the description and still be within the scope of the invention. Furthermore, obvious changes, modifications or variations will occur to those skilled in the art. Also, all references cited above are incorporated herein, in their entirety, for all purposes related to this disclosure.

What is claimed:

1. A method for treating a tumor that contains fibrin in a subject comprising:
(A) delivering energy from an energy source to the tumor, wherein the energy source comprises a source selected from the group consisting of acoustic energy source, electromagnetic energy source and light source; and
(B) administering to the subject a composition comprising a tumor-targeting monoclonal antibody in conjunction with a toxin.

2. The method of claim 1, wherein steps (A) and (B) are concurrently conducted.

3. The method of claim 1, wherein step (A) is conducted prior to the step (B).

4. The method of claim 1, wherein the energy is subcutaneously delivered to the subject using a probe.

5. The method of claim 1, wherein the energy is delivered to the subject through a body lumen using a catheter.

6. The method of claim 1, wherein the energy is sonic energy.

7. The method of claim 6, wherein the sonic energy is within the range from about 1,000 Hz to about 16,000 Hz.

8. The method of claim 1, wherein the energy is ultrasonic energy.

9. The method of claim 8, wherein the ultrasonic energy is within the range from about 16 k Hz to about 2 MHz.

10. The method of claim 8, wherein the ultrasonic energy is within the range from about 20 k Hz to about 1 MHz.

11. The method of claim 1, wherein the energy is radio frequency.

12. The method of claim 11, wherein the radio frequency is within the range from about 50 kHz to about 50 MHz.

13. The method of claim 11, wherein the radio frequency is within the range from about 500 kHz to about 10 MHz.

14. The method of claim 1, wherein the energy source is laser.

15. The method of claim 1, wherein the tumor-targeting monoclonal antibody is a monoclonal antibody that competitively inhibits the immunospecific binding of the MH1 antibody produced by hybridoma ATCC HB 9739 to MH1's target antigen, wherein said monoclonal antibody does not crossreact with: (a) fibrinogen, (b) plasmin derived fibrinogen degradation products and (c) plasmin derived fibrin degradation products.

16. The method of claim 1 which further comprises a step for detecting the tumor prior to delivering the energy.

17. The method of claim 16, wherein the detecting step comprises administering a composition comprising a monoclonal antibody targeting tumor in conjunction with a detectable marker.

18. The method of claim 1, wherein the tumor is a solid tumor.

19. The method of claim 1, wherein the tumor is sarcoma or carcinoma.

20. The method of claim 19, wherein the tumor is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, glioblastoma, astrocytoma, medulloblastoma, craniopharyngioma, ependymoma, pinealoma, hemangioblastoma, acoustic neuroma, oligodendroglioma, menangioma, neuroblastoma, and retinoblastoma colorectal adnoma, malignant melanoma, uveal melanoma, primitive neuroectodermal tumor, papillary carcinoma of the thyroid, alveolar rhabdomyosarcoma, plemorphic adenoma of salivary glands, sporadic typical lipomas, extraskeletal nyxoidchondrosarcoma, mucoepidemoid carcinoma, adenolymphoma of salivary gland, intraabdominal desmoplastic small round cell tumor, askins tumor, ethesioneuroblastoma, uterine leiomyomas, and myxoid liposarcoma.

21. The method of claim 1, wherein the tumor is a fibroid.

22. The method of claim 1, wherein at least a portion of the tumor is surrounded by the fibrin.

23. The method of claim 1, wherein the subject is human.

24. A method for eliminating tumor cells that associates with fibrin from a cell population that includes non-tumor cells that do not associate with fibrin comprising:
(A) applying energy from an energy source to the cell population, wherein the energy source comprises an energy source which is selected from the group consisting of an acoustic energy source, an electromagnetic energy source and a light source; and
(B) killing the tumor cells with a composition comprising a tumor-targeting monoclonal antibody in conjunction with a toxin.

25. The method of claim 24, wherein the energy is sonic energy.

26. The method of claim 25, wherein the sonic energy is within the range from about 1,000 Hz to about 16,000 Hz.

27. The method of claim 24, wherein the energy is ultrasonic energy.

28. The method of claim 27, wherein the ultrasonic energy is within the range from about 16 kHz to about 2 MHz.

29. The method of claim 27, wherein the ultrasonic energy is within the range from about 20 kHz to about 1 MHz.

30. The method of claim 24, wherein the energy is radio frequency.

31. The method of claim 30, wherein the radio frequency is within the range of from about 50 kHz to about 50 MHz.

32. The method of claim 30, wherein the radio frequency is within the range of from about 500 kHz to about 10 MHz.

33. The method of claim 24, wherein the energy is laser.

34. The method of claim 24, wherein the tumor-targeting monoclonal antibody is a monoclonal antibody that competitively inhibits the immunospecific binding of the MH1 antibody produced by hybridoma ATCC HB 9739 to MH1's target antigen, wherein said monoclonal antibody does not crossreact with: (a) fibrinogen, (b) plasmin derived fibrinogen degradation products and (c) plasmin derived fibrin degradation products.

35. The method of claim 24, wherein the non-tumor cells are hematopoietic stem cells.

36. The method of claim 24, wherein the tumor cells are derived from a solid tumor, sarcoma or carcinoma.

37. The method of claim 36, wherein the tumor cells are selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, glioblastoma, astrocytoma, medulloblastoma, craniopharyngioma, ependymoma, pinealoma, hemangioblastoma, acoustic neuroma, oligodendroglioma, menangioma, neuroblastoma, and retinoblastoma.

38. The method of claim 24, wherein at least some of the tumor cells are surrounded by fibrin.
